(19) United States
(12) Patent Application Publication
(10) Pub. No.: US 2010/0291038 A1
(43) Pub. Date: Nov. 18, 2010

(54) Novel Medicinal Formulation for Inducing an Immune Response in Patients with Chronic and Recurring Infections

(76) Inventor: Rajesh Shah, Maharashtra (IN)

Correspondence Address: BIO INTELLECTUAL PROPERTY SERVICES (BIO IPS) LLC, 8509 KERNON CT., LORTON, VA, 22079, US

(21) Appl. No.: 12/083,228

(22) PCT Filed: Feb. 21, 2007
(86) PCT No.: PCT/IN2007/000073

§ 371 Date: Apr. 1, 2010

(30) Foreign Application Priority Data
Jul. 6, 2006 (IN) 1063/MUM/2006

(51) Int. Cl. A61K 35/74; A61P 11/00

(52) U.S. Cl. 424/93.4

(57) ABSTRACT

A composition for the treatment of respiratory tract infection (RTI) comprising, a homogenized mixture of at least two serially diluted and potentized substances, as herein described, selected from: 1. Mycobacterium tuberculosis (culture) (strain A), 2. Mycobacterium bovis (culture) (strain B), 3. Mycobacterium microti (culture) (strain C), 4. Mycobacterium African (culture) (strain D), 5. Mycobacterium Lapre (from cultivation on the mouse footpad) (strain E), said dilution being effected in a vehicle selected from a group consisting of normal saline, distilled water and ethyl alcohol (90 to 100%).

### FIELD OF INVENTION

The present invention relates to a novel medicinal formulation for inducing an immune response in patients with chronic and recurring infections.

In particular, this invention relates to a novel medicinal formulation for prevention and treatment of various symptoms and conditions associated with Upper respiratory tract Infections [URTI], pre-tuberculosis, active tuberculosis and post tuberculosis states of infection and other chronic infections.

### DEFINITIONS

As used in the present specification, the following words and phrases are generally intended to have the meanings as set forth below, except to the extent that the context in which they are used indicates otherwise.

Antigenicity means the ability of a substance to trigger an immune response in a particular organism.

URTI means Upper Respiratory Tract Infections and affections. LRTI means Lower Respiratory Tract infections and affections. LTBI means Latent TB infection. For the purposes of this invention these infections are collectively referred to as respiratory tract infections [RTI]

URTI is a collection or combination of various infections including but not limited to Frequent Cold and cough, recurrent ear infections, allergic colds, tonsillitis, sinusitis, etc. LRTI includes recurrent bronchitis (infectious and allergic), Asthmatic bronchitis, bronchiolitis, bronchial asthma, and other pulmonary infections. Recurrent Upper and Lower Respiratory Tract infections may be associated with poor appetite (anorexia), inability to gain weight, loss of weight and Lean constitution.

Potency means capacity of the formulation to produce strong immunological response and/or defense.

### BACKGROUND AND PRIOR ART

Tuberculosis (commonly abbreviated as TB) is an infection caused by the bacterium Mycobacterium tuberculosis, which most commonly affects the lungs (pulmonary TB) but can also affect the central nervous system (meningitis), lymphatic system, circulatory system (Miliary tuberculosis), genitourinary system, gastrointestinal system, liver, eyes, bones and joints.

Most of those infected (90%) have asymptomatic latent TB infection (LTBI). There is a 10% lifetime chance that LTBI will progress to TB disease which, if left untreated, could kill more than 50% of its victims. TB is one of the top four infectious killing diseases in the world.

Although tuberculosis can generally be controlled using chemotherapy such treatment is not sufficient to prevent the spread of the disease. Furthermore, Potential hepatotoxicity of some of the first-line antitubercular agents remains a problem, especially during the initial period of treatment. There is always a risk of discontinuation by the patient because of side effects. For example, drugs like isoniazid, rifampin or pyrazinamide are sometimes discontinued by patients because of severe side effects like hepatotoxicity, exanthema and arthralgia.

Infected individuals may be asymptomatic, but contagious, for some time. In addition, compliance with the treatment regimen is critical. Some patients do not complete the course of treatment, which can lead to ineffective treatment and the development of drug resistant mycobacteria.

Several efforts have been made for the preparation of a vaccine for tuberculosis.

UK patent 2236480 describes tuberculosis vaccine. The UK patent describes vaccine to provide protection against tuberculosis.

An immunotherapeutic agent administered through parenteral route, consisting essentially of killed cells of Mycobacterium Vaccae, useful for treatment of Mycobacterial disease like tuberculosis or leprosy as an adjuvant to chemotherapy, is disclosed in U.S. Pat. No. 4,724,144.

Vaccine containing specific immunogenic portion of Mycobacterium Vaccae for nonspecific immune-response amplifier is described in U.S. Pat. No. 6,001,361.

WO03075825 describe the method of treatment of tuberculosis involving administration of a formulation prepared using Mycobacterium w, while manufacture of a pharmaceutical composition containing cells of Mycobacterium w along with a carrier in a single formulation is disclosed in WO03075824.

Furthermore, WO05042013, WO03089462, US Patent application 20030236393, WO0198460, WO0175096, WO0039301, US Patent 20010012888, U.S. Pat. No. 6,613,881, US Patent 20030147911, EP 1398379 describe the use of various proteins, peptides, non peptides derived from different strains of Mycobacterium for improving the immune response in patients with tuberculosis.

Currently, vaccination with live bacteria is the most efficient method for inducing protective immunity. The most common mycobacterium employed for this purpose is Bacillus Calmette-Guerin (BCG), an avirulent strain of Mycobacterium bovis.

However, the safety and efficacy of BCG is a source of controversy. The protective efficacy of vaccine against TB, bacille Calmette-Guerin (BCG) varies considerably from region to region—from around 80% protection to none. The protective effect of BCG, where observed, is mainly in neonates and children against non-infectious forms of primary disease, such as meningitis, but there is little or no protection against infectious, post-primary, pulmonary TB in adolescents and adults.

The BCG vaccine is sourced from attenuated tubercular organisms, which is supposed to protect against Mycobacterium Tuberculosis infection. Also, BCG cannot be administered over and again.

Other strains of mycobacterium like Mycobacterium vaccae, Mycobatectrium have been tried for immunotherapy for tuberculosis, and leprosy. However, there is little information on the efficacy of these preparations.

Furthermore, administration of a vaccine in people with weaker immune systems, such as the elderly, those on steroid therapy and those under significant stress can pose serious risk.

For centuries, homeopathic practitioners have suggested that serially agitated dilutions of infectious agents such as bacteria (called “nosodes”) are effective in the prevention of infectious disease.

A ‘nosode’ is a homeopathic remedy prepared from a pathological specimen. The starting material for preparation of a nosode can be blood, pus, any other body secretion or excretion, or even a diseased fragment of tissue, such as a growth. Rabies nosode, for example, starts with the saliva of a rabid dog and is then “potentized”.

The preparation of nosodes derives from homotoxicology, a type of homeopathic therapy created by Hans-Heinrich Reckeweg in Germany in the first part of 18th century.

Homeopathic medicine, since its inception under Hahneman at the beginning of the 19th century, follows the principle of “infinitesimals.” From this notion, the dosages of nosodes are in very minute and diluted forms. Thus, nosodes are not nearly as harmful as the untreated pathological product.

A ‘nosode’ is similar to an “oral vaccine” in the sense that its purpose is to “immunize” the body against a specific as well as related disease conditions. The major difference between a nosode and a vaccine is, of course, the extremely small quantity of antigenic material in a nosode.

In the case of nosodes from bacteria, viruses or diseased tissues, the preparation introduces the molecular imprints of possible antigens and other constituents of the pathological agent to the immune system. The working of the nosode is based on the fact that the immune system is induced to develop a defence mechanism which is effective against variety of antigens with this kind of molecular imprint, without being exposed to the virulence of the living agent.

Nosodes are also used as inter-current remedies in the treatment of chronic diseases. This is the most common use of nosodes in Homeopathic practice.

Presently available nosodes like Tuberculinum Bovinum, and Bacillinum, a maceration made from a tubercular sputum presumed to be containing antigens from limited strains of Mycobacterium and therefore they suffer from the drawback of providing limited immune protection. Besides, the current preparation is supposed to be sourced from the sputum of a patient who was clinically diagnosed with Tuberculosis (before the year 1930), when correct diagnostic procedures and laboratory tests were not available. Also, the correct preparation sourced from the sputum of suspected Tuberculosis patient, would probably have not some strain of the Tuberculosis organisms, in addition, would also have other organisms (uncertain and many), protein present in the sputum, and other known unknown tissues. IN other words, the current Tuberculinum nosode available in the market is of absolute uncertain origin and all unknown content.

In case of Bacillinum (another Tubercular nosode), which was prepared over years back from the ‘macerated Tuberculous lung’, where no bacteriological study is available; the source obviously must have been contaminated many unknown organisms as well as lung tissues!

Presently available nosodes like Tuberculinum Bovinum and Bacillinum are sourced from macerated lung tissues of the patient suspected to be suffering from Tuberculosis and from the sputum of such suspected patient. The preparations used presently are sourced from over 50 years old samples and, then prepared from the said sources. These sources are certainly unreliable, uncertain and unscientific.

The limitations with the present preparations are:

1. No one is sure of the exact source as bacteriological evaluation was not in practice in early 1940s.

2. No one has made any new preparations since then.

3. The preparations made from the infected lung tissues would obviously have many other organisms (mixed infections) as well as the diseased tissue itself.

4. The strain of Mycobacterium organism was never available, as it was never investigated in the light of current understanding.

5. If at all they contained some Mycobacterium Tuberculosis organisms, the strain of such organisms have evolved a great deal in last six decades.

The old preparations as well as the presently available preparations, thus, present many uncertainties, unscientific nature of the source, non-reproducibility, and limited antigenicity and hence a major drawback of providing limited immune protection.

Therefore, there is a need for a formulation that can induce protective immunity against a broad spectrum of antigens related to tuberculosis and other chronic infections associated with tuberculosis as well as allergic infections of the respiratory system, in a re-producible manner and which has better safety profile than vaccines.

It is an object of this invention to provide a novel, broad spectrum immunity enhancing, reliable medicinal formulation prepared from the range of culture of the specific organisms, so as to prevent and cure various conditions, usually prevalent among patients with active or inactive tuberculosis infection and other chronic affectations, particularly, RTI. Interesting application of the new invention is that it works as prophylactic against a wide range of non-tubercular infections and allergies, probably by enhancing general immune status. It may be noted that the said preparation is not meant to be prescribed as a vaccine against Tuberculosis.

Yet another object of this invention is to provide a therapeutic measure for the treatment of various disease states, which may be present by one or more of the following indications:
1. Recurring cough and cold
2. Recurring fever
3. Loss of appetite
4. Malnourished state of health
5. Loss of weight due to any chronic infection (or due to any unknown reason) or an inability to gain weight
6. Recurring respiratory allergies and secondary infections in asthmatics, etc.
7. Shortening the span of recovery phase in case of any subacute infection such as Pneumonia, Septic tonsillitis, Liver abscess, tuberculosis of any organ, etc.
8. Enhancing general health and immunity in those who may have past or family history of Tuberculosis.

Yet another object of this invention is to provide a method for preparation of a novel formulation, potentized by means of a mathematico-mechanical process for maximal therapeutic benefits.

Yet another object of this invention is to provide a novel medicinal formulation which is reproducible and which contains precisely defined antigenic material.

Yet another object of this invention is to provide a novel medicinal formulation offering better patient compliance and thereby minimizing the risk of multi drug-resistant type of infections.

What is envisaged in accordance with this invention is a novel medicinal formulation with a mixture of antigens from killed cells of Mycobacterium of different strains namely, Mycobacterium tuberculosis, Mycobacterium bovis, Mycobacterium microti, Mycobacterium African and Mycobacterium laprae.

Particularly envisaged in accordance with this invention is a safe medicinal formulation containing serially agitated dilutions of mixture of antigens from aforementioned strains of Mycobacterium.

### SUMMARY

The composition of this invention referred to as Mycobacterium complex is found effective as a preventive measure against recurring infections, especially that affects the upper and lower respiratory tracts.

There is no such known therapeutic measure comparable with the Mycobacterium complex, known which has specific role in controlling the infections of the respiratory tract.

The exact mechanism of its action required to be explored. However, Mycobacterium complex seems to be working by enhancing immunity against the respiratory infections. This may broadly be compared with principle of vaccination.

However, the Mycobacterium complex has been found to be effective against a wide range of Non-Tubercular infections. Mycobacterium complex can administered many times, without any adverse effects.

Mycobacterium complex also helps gaining weight by controlling infections, correcting metabolism, enhancing appetite.

### Description

### Mycobacterium Complex:

Mycobacterium complex includes a wide range of Mycobacterium group of bacilli, inclusive of typical and atypical variants.

Mycobacterium is a genus of Actinobacteria, given its own family, the Mycobacteriaceae. All mycobacteria are aerobic and acid fast.

Cell walls of Mycobacteria are hydrophobic, waxy and rich in mycolic acids/mycolates and they are thicker than in many other bacteria,.

The genus Mycobacterium includes many pathogens known to cause serious diseases in mammals, including tuberculosis and leprosy they are also capable of affecting most organs in the human body such as lungs, skin, bones, brain, intestines, liver, eyes, kidney, etc. Some of the most common variants are:

1. Mycobacterium tuberculosis (culture) (strain A)
2. Mycobacterium bovis (culture) (strain B)
3. Mycobacterium microti (culture) (strain C)
4. Mycobacterium African (culture) (strain D
5. Mycobacterium Lapre (from cultivation on the mouse footpad) (strain E)

Cultures of the organisms numbered 1 to 4 in the above list are procured separately from a reputed Pathological Laboratory who regularly deal with bacterial cultures. Various strains of organisms (1 to 4) when detected in the sputum or other diseased tissues are cultured for specific strains.

Colonies of the specific organisms form the culture media are carefully isolated with a probe and put in normal saline for further processing. About 10 ml of each of the saline containing the specified organism from cultures are mixed separately in 20 ml normal saline. These are called as Primary culture. Thus 4 Primary cultures for Mycobacterium strains A, B, C, and D are prepared and are labeled as A_{PC}, B_{PC}, C_{PC}, D_{PC}.

In case of Mycobacterium Laprae, Mycobacterium leprae organism grown on armadillo, separated and centrifuged, with residues of some tissues (100% wash not possible), containing millions of live organisms (over 10⁻⁸ (bacteria) per ml is used for making a Primary culture. This was sources from a leading Leprosy research institute of India, located in Mumbai.

About 2 ml of the Mycobacterium leprae culture is taken in a sterile test tube and mixed with 2 ml of ethyl alcohol (90 to 100%) to kill the organisms to obtain E_{PC}. The killed Mycobacterium leprae organisms are known to retain antigenicity.

The organisms are found in the secretions of infected tissues.

Mycobacterium tuberculosis (culture) (strain A) was obtained from Ankur laboratory, of a patient from whose sputum was positive for AFT (Mycobacterium Tuberculosis), wherefrom the organisms were then cultured. Similarly, Mycobacterium bovis (culture) was obtained from a veterinary hospital pathology laboratory. (The bovine version is found to infect cattle. Mycobacterium microti (culture) (rodents and rarely humans) and Mycobacterium African cultures were similarly obtained.

Mycobacterium Leprae (from cultivation on the mouse footpad) was obtained from Leprosy Institute, Mumbai. Mycobacterium leprae organism grown on armadillo, separated and centrifuged, which included some tissues (100% wash not possible), which contained millions of live organisms (over 10⁻⁸ (bacteria) per ml. About two ml was taken in sterile test tube and mixed with 2 ml of ethyl alcohol to kill the organisms. The killed organisms are known to retain antigenicity.

The said preparation containing killed M. leprae bacteria was mixed in 2 ml: 90 drops of alcohol to undergo potentization, to arrive to M.L. 1c potency, for subsequent use.

It may be noted that homeopathic preparation in a potency higher than 14 c are found to have no physical traces or any molecules of the original source. In other words, all preparations after 15c are free from toxic properties of the original substances.

Thus, 5 Primary cultures for 5 strains of Mycobacterium labeled as A_{PC}, B_{PC}, C_{PC}, D_{PC} and E_{PC} are prepared. About (2 ml) of Primary culture (2 ml) is mixed with a vehicle selected from a group of vehicles consisting of normal saline, distilled water and ethyl alcohol(90 to 100%). Preferably, ethyl alcohol (90 to 100%) is used as a vehicle.

The proportion of mixing of primary culture with the vehicle can range from 1:99 to 50:50. Preferably 2 ml of the primary culture is mixed with 90 ml of the vehicle.

Although the dilution represented by the term ‘c’ means a dilution of 1:99, for the purposes of this specification the term ‘c’ is deemed to mean any dilution in the range of 1:99 to 50:50

### Serial Dilution and Potentization:

Potentization is a mathematico-mechanical process for rendering inert or poisonous antigen containing pathological residues, to a state of physical solubility, physiological assimilability so as to enhance their therapeutic activity and harmlessness, for use as a healing remedy.

The primary object of potentization is to reduce all substances designed for therapeutic use to “a state of approximately perfect solution or complete ionization, which is fully accomplished only by infinite dilution.” (Arrhenius.)

Each resulting diluted culture is potentization typically by stroking by holding the bottle in a closed fist and striking the fist on a hard surface repeatedly at a regular frequency or by exercising similar powerful stroke using a mechanical device which can strike a bottle on a hard surface. Such strokes are given about 10 times. This preparation is labeled as Strain 1c potency. Thus 5 different preparations of 1c are obtained and labeled as A-1c, B-1c, C-1c, D-1c and E-1c.

In accordance with the preferred embodiment of this invention, the bottle containing the preparation for stroking for potentization is a securely stoppered glass bottle and a mechanical device is adopted to exert a force of at least 6 dynes rhythmically at a frequency of 10 strokes in two minutes.

This procedure of serial dilution followed by stroking is repeated 4 times with each 1 c potency preparations to obtain respective 5c preparations for each strain. These are thus labeled as A-5c, B-5c, C-5c, D-5c and E-5c.

### Preparation of Mycobacterium Complex:

At this point, about 2 ml liquid from A-5c along with 2 ml liquid taken from at least one 5c preparation selected from a group of 5c preparations consisting of B-5c, C-5c, D-5c and E-5c is taken in a separate bottle and about 70 to 100 drops of vehicle are added to obtain different Mycobacterium Complex preparations such as Mycobacterium Complex AB 5c, Mycobacterium Complex AC 5c, Mycobacterium Complex AD 5c, Mycobacterium Complex AE 5c, Mycobacterium Complex ABC 5c, Mycobacterium Complex ABD 5c, Mycobacterium Complex ABE 5c, Mycobacterium Complex ACD 5c, Mycobacterium Complex ACE 5c, Mycobacterium Complex ADE 5c, Mycobacterium Complex ABCD 5c, Mycobacterium Complex ABCE 5c, Mycobacterium Complex ABDE 5c, Mycobacterium Complex ACDE 5c and Mycobacterium Complex ABCDE 5c.

A vehicle is selected from a group of vehicles consisting of normal saline, distilled water and ethyl alcohol. Preferably ethyl alcohol (90 to 100%) is used as a vehicle.

Above mentioned Mycobacterium complex 5c preparations thus obtained are then further subjected to serial dilutions and potentization to obtain preparations with higher potencies like M.C.-6c, M.C.-7c, M.C.-8c . . . M.C.-50c . . . M.C.-1000c. M.C.-50000c and above.

Advantage and Application of the new preparation Mycobacterium complex 6c to 100000c (and above):

These preparations are prepared from the specific bacterial strains but they lack the bacterial toxicity.

Preliminary clinical investigations have shown that the formulations prepared according to the above process help to retain the capacity to induce immune response in the body, which helps in prevention and treatment of various conditions, such as recurring upper and lower respiratory infections and allergies, recurring viral infections, Anorexia (loss of appetite), weight loss or not gaining weight.

Furthermore, they positively alter the course of any form of tuberculosis and are useful for preventing, controlling or treating all sorts of opportunistic infections commonly found in cases of Tuberculosis as well as other chronic infections such as AIDS.

Mycobacterium tuberculosis (culture) (strain A) was obtained from Ankur laboratory, of a patient from whose sputum was positive for AFT (Mycobacterium Tuberculosis), wherefrom the organisms were then cultured. Similarly, Mycobacterium bovis (culture) was obtained from a veterinary hospital pathology laboratory. (The bovine version is found to infect cattle. Mycobacterium microti (culture) (rodents and rarely humans) and Mycobacterium African cultures were similarly obtained.

The organisms were found in the secretions of infected tissues.

Mycobacterium Lapre (from cultivation on the mouse footpad) was obtained from an eminent Leprosy institute in Mumbai. Mycobacterium leprae organism grown on armadillo, separated and centrifuged, which included some tissues (100% wash not possible), which contained millions of live organisms (over 10⁻⁸ (bacteria) per ml. About two ml was taken in sterile test tube and mixed with 2 ml of ethyl alcohol to kill the organisms. The killed organisms are known to retain antigenicity.

The said preparation containing killed M. leprae bacteria was mixed in 2 ml: 90 drops of alcohol to undergo potentization, to arrive to M.L. 1c potency, for subsequent use.

### About the Potency:

MTC was prepared in a range from MTC 1c to MTC 100000c, where ‘c’ denotes centesimal potency. The medicines prepared from the diseased body tissues or organisms are prescribed to patients in various potencies, as per the well defined parameters of potency selection. Some of the parameters may be described in brief hereunder:

1. Age: Younger patients with higher susceptibility may be prescribed medium to high potency such as 30c to 1000c.

2. Functional pathology: Patients with functional disorders are prescribed medium to high potency.

3. Structural pathology: Patients with structural pathology are prescribed low potency to start with.

4. Nosode: When used as a nosode, 30c potency is the ideal in it can be stepped up slowly.

5. Response: In case there is no response or the remedy stops acting, the potency can be stepped up from 30c to 50c to 100c to 500c.

6. Active pathology: In case of active pathology such as tubercular cavities or ulcers, higher potencies should be avoided.

7. Susceptibility: Higher the susceptibility, higher the potency.

### REFERENCES

1. J. T. Kent's Philosophy
2. M. L. Dhawale's Principles and Practice of Homoeopathy

It may be noted that homeopathic preparation in a potency higher than 14c are found to have no physical traces or any molecules of the original source. In other words, all preparations after 15c are free from toxic properties of the original substances.

Some case illustrations of Mycobacterium Tuberculosis Complex (MTC)

### Case I: Diagnosis: Recurrent Cough and Cold.

17 years old male, reported, with the complaint of Recurrent Cough and cold, since three years. He would get an episode once or twice in a month, lasting for about seven days. He was required to take antibiotics every month for last five months.

He was prescribed M.C.T. with a combination of Mycobacterium Complex ABCD , 30c; one dose a week for one month. He was reviewed every month.

Report: No episode of cough and cold since starting the treatment.

### Case II: Diagnosis: Recurrent Colds

A female child, 5 years old, reported with the complaint of Recurrent colds, once every month lasting for about 10 days, requiring conventional treatment with cough syrup and antibiotics. She was not putting on weight. Weight 15.5 kg.

She was prescribed Tuberculinum 1m, infrequent doses, once a month for three months. (Tuberculinum 1m is a traditional homeopathic nosode prepared from the lung tissues presumed to be infected with tubercular infection. There was not improvement in the frequency of colds nor any weight gain after three months. Her weight was 21 kg. Patient discontinued the medicine, took conventional medicines during this period, only to get temporary relief. She reported again.

M.C.T Mycobacterium Complex ABCE30c, one dose a month was prescribed and subsequently. Case was reviewed every month and concluded after three months. She had only one episode of cold thereafter. She gained two kilograms of weight and she maintained good health. She was prescribed M.C.T. Mycobacterium Complex ABCE , 80c, M.C.T. Mycobacterium Complex ABCE 100c, and M.C.T. Mycobacterium Complex ABCE, 120c one dose each, every month. Her case was reviewed again after a year. The patient had had no episode of colds at all during this period. She gained weight by two more kilograms, when last weighed was 25 Kg.

Case III: Diagnosis: Recurrent Cough and Cold (Rec. URTI)

Three years old child, who was born 20 days premature, was brought for the treatment, with the complaint of Recurrent Upper Respiratory Tract Infections; once every month, lasting for seven days (with antibiotics) or longer if no medicines taken. He would start with colds, rhinorrhea and then develop chest congestion with cough and expectoration; along with fever. He was weighing 8 Kg and was not putting on weight. He had delayed milestones.

He was prescribed Tuberculinum 1m, once a month for four months with partial relief.

Patient reported again (after 15 months) with sub-acute complaint of cough and cold (bronchitis) for 10 days. He was prescribed M.C.T Mycobacterium Complex ABCDE , 30c twice a day for two weeks. His cough-cold and appetite improved. He was put on M.C.T 30c, twice a day for three more weeks, and again on M.C.T 50c and MCT 60c for two subsequent months. He improved significantly and steadily.

After 6 months, he was free from attacks of cough and cold, his weight was 15 Kg, having gained seven kilogram.

### Case IV: Diagnosis: Recurrent Cough and Cold

Sixty seven years old gentleman approached us for the treatment of Recurrent cough and cold since 30 to 40 years. He had cold and cough almost daily, for which he had taken allopathic medicines as well as homeopathic medicines on many occasions, but nothing helped him to control his recurring problem. He also has sense of weakness, frequent headache and sleeplessness for many years.

He was given Tuberculinum 1m without any relief. Later, he was prescribed MCT complex ABCDE 30, one dose. He reported 50% improve in his cough and cold (acute state), as well as his general health and sleep. M.C.T 30c, M.C.T. 80c, M.C.T. 100c were prescribed in increasing dose once a month.

His frequent colds and cough of over 30 years duration improved by over 70% with significant improvement in his general health and sleep pattern.

### Case V: Diagnosis: Recurrent Upper and Lower Respiratory Tract Infections

Five years old female child was referred for the treatment by a homeopath colleague. The child presented with Recurrent Upper and Lower Respiratory Tract infections, in the form of colds, cough, chest infection, fever; with low energy, ‘always tired’ and no weight gain. The child was prescribed Tuberculinum 1M, Silica 200, Calcaria Phosphorica 200, etc. by the referring homeopath, without much improvement.

She was prescribed M.C.T. ABCDE 30c one dose and repeated once a month for six months. The frequency of Upper and Lower respiratory tract improved by over 50%, with improvement in sense of well being. The treatment was continued, M.C.T 30c to 100c, one year with remarkable improvement. The treatment was concluded.

Case V: Diagnosis: Recurrent Cough and Cold with Asthmatic Bronchitis.

Three and half years old made child was brought with the complaint of Recurrent cough and cold since last three and half years; once every month lasting for 10 to 20 days; with episodes of Asthmatic bronchitis (wheezing). He was given steroids every month by the paediatrician for the wheezing with little relief.

He was prescribed M.C.T. ABCDE 30c one dose once a month, for about four months.

He reported significant reduction in the frequency, severity and duration of the attacks of Cough, Cold and wheezing; required much less courses of antibiotics and steroids.

### Case VI: Diagnosis: Tuberculosis of Kidney

Sixty four years old female presented from long distance (Ahmedabad) Kochs Kidney, with history of no response to the conventional Anti Tubercular line of treatment (R-cin) for 10 months. She had fever every day for last 14 months, tired feeling, and low energy. Her ERS was 121.

She was prescribed M.C.T ABCDE 30C THREE doses, every week, without any relief. She was prescribed M.C.T 30c twice a day for one month. After 4 months, she reported significant improvement in general health, and reduction in fever. Her E.S.R. was 110. Next month, she reported no further improvement in her condition and she was suggested to go for another course of AKT.

Conclusion: M.T.C. may have supportive role in the cases of active tuberculosis. It may or may note work as anti-tuberculosis measure. Further exploration is called for.

Case VII: Diagnosis: Recurrent Cough and Cold with Asthmatic Bronchitis

three years old female child was brought with the complaint of Recurring Cough (once in 10 days), cold (once in 10 days) and asthmatic breathing (once in 15 days) since two years. She had poor appetite and no weight gain.

She was prescribed M.T.C. ABCDE 30c, two doses which were repeated every month for one year. Her attacks of colds, cough and asthma almost stopped, with improvement appetite and weight gain.

### Case VIII: Diagnosis: Recurrent Cough and Cold

Three years, eight months old male child presented with Recurrent Cough and Cold since birth. Frequency: Once in 10 to 15 days, lasting for about seven days.

He was prescribed M.T.C ABCDE 3oc. one dose once a week, for about eight weeks. His treatment was concluded as he was free from frequent attacks of his chief problems. He gained weight and improved his general health.

### Case IX: Diagnosis: Recurrent Cough and Cold

Male child, 1 year eight months, brother of above patient presented with similar problems of Recurrent cough and cold since one year. Episodes once a month, lasting for about 10 days with the convention medicines.

M.T.C. ABCDE 30c and then M.T.C. ABCDE 50c, one dose a forth night, for about five months, were prescribed. The child was almost free from frequent attacks of Upper and lower respiratory tract infections.

### Case X: Diagnosis: Recurrent URTI

Ten years old female child was brought with the complaint of Recurrent URTI since last 3 to 4 years, more or less daily attacks of nasal catarrh. She also used to breath through mouth due to chronic nasal congestion.

She was prescribed MTC ABCDE 30c, one doses. She reported on the third month with improvement of about 75% in all her complaints.

Thus MTC is a medicinal preparation is useful

Frequent Cold and cough

Recurrent tonsillitis

Recurrent sinusitis

Asthmatic bronchitis

Poor appetite

Lean constitution, not gaining weight

Recurrent upper respiratory tract infections

Recurrent lower respiratory tract infections

Sinusitis: acute, chronic, recurrent

Tonsillitis: acute, chronic, recurrent

Asthmatic bronchitis

Anorexia

### Clinical Trials:

MTC was used in a sample study of 162 patients out of these cases, 114 had recurrent URTI, 20 cases had recurrent tonsillitis, 16 had complaints of a lean constitution and not gaining weight and 12 cases with complaints of recurrent cough. Improvement has been observed in less than eight weeks in all the cases and the cases have been followed up for over a year. The success rates are tabulated below:

| | No. of | Improvement | | Improvement | | Less than 50% | | No | |
|---|---|---|---|---|---|---|---|---|---|
| Condition | cases | 75-99% | | 50-75% | | improvement | | Improvement | |
| Recurrent | 114 | 80 | cases (71%) | 21 | cases (18%) | 5 | cases (4.3%) | 8 | (7%) |
| URTI | | | | | | | | | |
| Recurrent | 20 | 13 | cases (65%) | 3 | cases (15%) | — | | 4 | (20%) |
| tonsillitis | | | | | | | | | |
| Loss of appetite | 16 | 12 | cases (75%) | 1 | case (6%) | 2 | cases (9%) | 1 | (9%) |
| and low weight | | | | | | | | | |
| Recurrent cough | 12 | 7 | cases (60%) | 3 | cases (25%) | — | | 2 | (15%) |
| Total cases | 162 | 112 | (69%) | 28 | (17%) | 7 | (4.5%) | 15 | (9.5%) |

Dose: MTC is administered in tablet form, to be taken one dose a day for six weeks. The course may be repeated after 10 weeks.

Side effects and interaction: No adverse effects were observed in any of the 162 cases.

In most cases, improvement was observed in about four weeks of usage. Frequency of attacks (recurrent RTI) starts reducing after about four weeks of use. Appetite improves in about three week time. Weight gain was observed in about four to eight weeks in most cases.

While considerable emphasis has been placed herein on the specific steps of the preferred process, it will be appreciated that many steps can be made and that many changes can be made in the preferred steps without departing from the principles of the invention. These and other changes in the preferred steps of the invention will be apparent to those skilled in the art from the disclosure herein, whereby it is to be distinctly understood that the foregoing descriptive matter is to be interpreted merely as illustrative of the invention and not as a limitation.

1. A composition for the treatment of respiratory tract infection (RTI) comprising, a homogenized mixture of at least two serially diluted and potentized substances, as herein described, selected from:
1. Mycobacterium tuberculosis (culture) (strain A)
2. Mycobacterium bovis (culture) (strain B)
3. Mycobacterium microti (culture) (strain C)
4. Mycobacterium African (culture) (strain D
5. Mycobacterium Lapre (from cultivation on the mouse footpad) (strain E), said dilution being effected in a vehicle selected from a group consisting of normal saline, distilled water and ethyl alcohol(90 to 100%).

2. A composition as claimed in claim 1, wherein the Mycobacterium Lapre is obtained by killing live Mycobacterium Lapre, in ethyl alcohol.

3. A composition as claimed in claim 1, wherein each of the serially diluted substances are 5c dilutions of the primary cultures of each of the strains in the vehicle, each ‘c’ representing a dilution of the previous stage in the vehicle where the proportion of the culture to vehicle is in the range of 1:99 to 50:50.

4. A composition as claimed in claim 1, wherein the serial dilution is in the range of 6c to 100000c.

5. A composition as claimed in claim 1, wherein the serial dilution is 30 to 200c.

6. A process for making a composition as claimed in claims 1 to 5, comprising the following steps
i. obtaining Mycobacterium tuberculosis (culture) (strain A)
ii. obtaining Mycobacterium bovis (culture) (strain B)
iii. obtaining Mycobacterium microti (culture) (strain C)
iv. obtaining Mycobacterium African (culture) (strain D
iv. obtaining Mycobacterium Lapre (from cultivation on the mouse footpad) (strain E);
v. preparing primary cultures of strain A, strain B, strain C and strain D by diluting solutions containing the culture with about twice the quantity of a vehicle selected from a group consisting of normal saline, distilled water and ethyl alcohol(90 to 100%) and preparing primary culture of Strain E by killing live Mycobacterium Lapre with ethyl alcohol and diluting the so obtained solution with twice the quantity of a vehicle to obtain a primary culture of strain E;
vi. serially diluting primary cultures of strains A, B, C, D and E with the vehicle in a ratio of culture to vehicle in the range of 1:99 to 50:50 to obtain a 5c stage dilution of each of the Strains A, B, C, D, and E with potentization at each stage;
vii. mixing the 5c dilutions of at least two of the strains selected from strains A, B, C, D and E to obtain a Mycobacterium primary complex; and
viii. serially diluting with potentization, the Mycobacterium primary complex with the vehicle in a ratio of complex to vehicle in the range of 1:99 to 50:50 to obtain diluted Mycobacterium primary complex of 6c to 100000c.

7. A process for making a composition as claimed in claim 6, wherein potentization is effected by stroking by holding a bottle containing the diluted culture in a closed fist and striking the fist on a hard surface repeatedly at a regular frequency or by exercising similar powerful stroke using a mechanical device which can strike a bottle on a hard surface.

8. A process for making a composition as claimed in claim 7, wherein strokes are given about 10 times.

9. A process for making a composition as claimed in claim 7 or 8, wherein the bottle containing the preparation for stroking for potentization is a securely stoppered glass bottle.

10. A process for making a composition as claimed in claimed in claims 7 to 9 in which a mechanical device is used to exert a force of at least 6 dynes rhythmically at a frequency of 10 strokes in two minutes.
